SEQUENCE LISTING (3 sequences)

SEQ ID NO: 1 - DNA, 1549 bp, artificial sequence
  DNA sequence of the S2-6 gene
gtcagcaagaggtgtggcatgtttgggatgcaaggggacgtgttcgggcttcgagcacattcatggaggaaaatatgcaagtcttgcaaatgcagccaagaggaccactgcctaacatctgacctagaagacgatcggaaaattggccgcttgctgatggactccaagtattccaccctcactgctcgggtgaaaggcggggacggcatccggatttacaagaggaaccggatgatcatgaccaaccctattgctactgggaaagatcccacttttgacaccatcacctacgagtgggctccccctggagtcacccagaaactgggactgcagtacatggagctcatccccaaggagaagcagccagtgacaggcacagagggtgccttttaccgccgccgccagctcatgcaccagctccccatctatgaccaggatccctcgcgctgccgtggacttttggagaatgagttgaaactgatggaagaatttgtcaagcaatataagagcgaggccctcggcgtgggagaagtggccctcccgggcagggtggcttgcccaaggaggaggggaagcagcaggaaaagccagagggggcagagaccactgctgctaccaccaacggcagtctcagtgacccgtccaaagaagtggaatacgtctgcgagctctgcaagggagcggcccctcctgacagccccgtggtctactcgacagggcaggctacaacaagcagtggcaccccacctgctttgtgtgtgccaagtgctccgagccgctggtggacctcatctacttctggaaggatggtcaccctggtgcggccgccattactgcgagagtctgcggccccggtgctccggctgcgatgagataatattcgctgaggactaccagcgtgtggaagatctggcctggcaccgaaagcactttgtctgtgagggttgtgagcagctgctgagcggccgggcgtacatcgtcaccaagggtcagcttctgtgcccaacttgcagcaagtccaaacgctcctgaagggctgcccacccacagccagaatccacaggatcccaccgagaaggagccaggtgtgccgagaccatcctaagggtccgatgtgacagcaagcaagtgaataaacaatgatttgcttttcagtgagaatatatatatgagatatatatagatatatatctaggttgggtggtggtagatccttgagggtcagtagtttcaaaaccaaaaatattctaagaagtcttaggatggagttccttttctttctgttgttgtttcccagctacaaccaactaaagacacaaatggcgttctgcaaggggactctgggaggagttttccagaatgcaattccgagtgagcaaatcgcatagctgtagaatgtgcgtgcttttttgtggacacaggagctcctccaggagcaggctgggatcccaactatcgcttgttgcctctttttcaagtggaatttgaattttaaataaacaactttttttggcatgataaacagatcaataaaagttttgtgaattcc

SEQ ID NO: 2 - protein, 343 aa, artificial sequence
  Amino acid sequence of S2-6 protein
VSKRCGMFGMQGDVFGLRAHSWRKICKSCKCSQEDHCLTSDLEDDRKIGRLLMDSKYSTLTARVKGGDGIRIYKRNRMIMTNPIATGKDPTFDTITYEWAPPGVTQKLGLQYMELIPKEKQPVTGTEGAFYRRRQLMHQLPIYDQDPSRCRGLLENELKLMEEFVKQYKSEALGVGEVALPGRVACPRRRGSSRKSQRGQRPLLLPPTAVSVTRPKKWNTSASSARERPLLTAPWSTRQGRLQQAVAPHLLCVCQVLRAAGGPHLLLEGWSPWCGRHYCESLRPRCSGCDEIIFAEDYQRVEDLAWHRKHFVCEGCEQLLSGRAYIVTKGQLLCPTCSKSKRS

SEQ ID NO: 3 - protein, 52 aa, artificial sequence
  domain 286..337: Amino acid sequence of the zinc binding LIM domain of S2-6
CSGCDEIIFAEDYQRVEDLAWHRKHFVCEGCEQLLSGRAYIVTKGQLLCPTC

(19) United States
(12) Patent Application Publication
(10) Pub. No.: US 2004/0005646 A1
(43) Pub. Date: Jan. 8, 2004

(54) Use of the S2-6 gene product as a diagnostic marker for cancer detection

(76) Inventors: Beata Lecka-Czernik, Little Rock, AR (US); Lisa Greene, Houston, TX (US); William Gallagher, Houston, TX (US)

Correspondence Address: Benjamin Aaron Adler, ADLER & ASSOCIATES, 8011 Candle Lane, Houston, TX, 77071, US

(21) Appl. No.: 10/396,260

(22) Filed: Mar. 25, 2003

Related U.S. Application Data
(60) Provisional application No. 60/367,589, filed on Mar. 26, 2002.

(51) Int. Cl. G01N 33/574

(52) U.S. Cl. 435/7.23

(57) ABSTRACT

Provided herein are methods of detecting a cancer or a pre-cancer in a subject. In these methods the amount of antibody or antibody fragment bound to an epitope of a S2-6 DNA binding protein or, alternatively, the amount of S2-6 mRNA expression in a first tissue sample from the subject suspected of being cancerous or precancerous is compared with the levels of binding or expression, respectively, in a second sample taken from healthy tissue as the same type as the first sample. No binding or a lesser binding of the antibody or antibody fragment, or, alternatively, no S2-6 mRNA expression or lesser expression by the first sample as compared to that of the second sample is indicative of a cancer or a pre-cancer in the subject. Also provided are methods of treating a cancer or a pre-cancer in a subject. Kits are provided to perform the methods disclosed herein.

### CROSS-REFERENCE TO RELATED APPLICATION

This non-provisional patent application claims benefit of provisional patent application U.S. Serial No. 60/367,589, filed Mar. 26, 2002, now abandoned.

### BACKGROUND OF THE INVENTION

1. Field of the Invention

This invention relates generally to molecular biology and the methods of cancer detection. More specifically, the present invention relates to the use of S2-6 gene products for early cancer detection.

2. Description of the Related Art

Tumorigenesis is considered to be primarily a multi-step process that leads to the progressive transformation of normal cells into malignant derivatives. The earliest hallmark of tumorigenesis is the re-entry of non-dividing, usually post-mitotic, cells into the cell cycle. This process is due to escape of normal cells from the controlling mechanisms that regulate cell division. Enhanced expression of genes and gene products that promote cell proliferation in cancerous cells allows these cells to increase in number.

The chances of effectively treating many cancer subtypes are in direct relation to the developmental staging of the disease. For example, the 5-year survival rate of patients with colorectal cancer is 92% if detected in the early stages, however, only 37% of these cancers are detected at this stage.

Many detection methods rely on the visualization of abnormal tissue via various imaging techniques, e.g., sigmoidoscopy or colonoscopy for colon cancer, ultrasound for ovarian cancer and mammography for breast cancer. However, at times, to be detectable in this manner, the tumor has advanced to a stage precluding early stage therapies; indeed, often diagnosis is made at the instigation of the patient who has either detected an abnormality or is experiencing symptoms of the disease. Thus, earlier detection of cancer should lead to considerable improvement in terms of patient response rates and overall survival.

Diagnostic methods have been developed that utilize genetic markers or gene products specific to a cancer. U.S. Pat. No. 6,284,241 details the immunodiagnosis of colon cancer using polypeptides that comprise an immunogenic portion of a colon tumor protein or a variant of the protein. An immunoassay detecting the presence of the colon tumor protein in a biological sample from the patient indicates the presence of colon cancer. However, this assay is applicable to only colon cancer.

U.S. Pat. No. 6,329,505 discloses methods and compositions for the diagnosis of prostate cancer. These compositions may be prostate-specific proteins, immunogenic portions thereof, or polynucleotides that encode such portions and are detected by standard immunoassay, PCR or hybridization techniques. Expression levels are compared to a pre-determined cut off point determined from an average of expression levels in patients without prostate cancer.

U.S. Pat. No. 6,251,586 discloses an epithelial protein, peptide and variants of such whose presence in an epithelial cell indicates the presence of a precancer, particularly lung, breast, bone, ovary, prostate, kidney, melanoma and myeloma. Computer imaging techniques are used to screen for cells expressing hnRNP mRNA and to generate a method of computer diagnosis of cancer.

U.S. Pat. No. 6,297,172 discloses methods of detecting tumor-associated antigens of breast, ovaries, colon, pancreas or lung, particularly the pancreas, by binding the monoclonal antibody 494/32-DSM ACC 2410 or monoclonal antibody 495/36-DSM ACC 2415 to the antigen and measuring the amount of the binding. In vitro binding is measured using an indirect immunofluorescence assay, inhibition of enzyme release, or inhibition of pinocytosis of colloidal gold; in vivo binding measurements are performed by immunoscintigraphy.

U.S. Pat. No. 6,218,529 discloses nucleic acid sequences useful as oligonucleotide hybridization probes and primers that hybridize selectively to specific markers of prostate cancer, BPH, bladder cancer or breast cancer. These probes and primers are useful for the diagnosis, identifying and monitoring of these cancers through the measurement of gene products.

U.S. Pat. No. 5,959,081 identified and characterized a polypeptide, designated S2-6. The S2-6 gene (ATCC NO. 97642), originally isolated from cells undergoing a process of premature senescence, codes for a protein which contains a specific zinc binding LIM domain (Group 2 of the LIM family). The LIM protein family has roles in developmental and cell growth regulation. S2-6 is overexpressed in nonproliferating or growth inhibited human diploid fibroblasts, but expression is abolished or significantly diminished in fetal human diploid fibroblasts, immortalized or tumor derived cell lines, and other highly proliferative cells. Because the protein is absent in highly proliferating cells, it could be used as a marker for cancer detection.

However, the expression of a potential cancer marker in vitro does not necessarily correlate with in vivo expression. For example, the p21 (CIP1/WAF1) gene could not be used as a marker for a diagnostic test for cancer. p21 mRNA and protein levels are highly elevated in vitro in growth arrested cells due to senescence, DNA damage, or growth conditions and p21 expression is decreased in proliferating normal and immortal cells (1-3); its expression, however, is not decreased in cancer cells in vivo (4-6) and, therefore, is useless as a marker for cancer detection.

Thus, as S2-6 is expressed in vivo, strategies of overexpressing S2-6 protein in cancerous cells may lead to inhibition of cell growth that is important in anti-cancer therapy. The inventors have recognized an additional need in the art for an in vivo diagnostic method of early cancer detection using a genetic marker whose applicability is not restricted to a specific cancer. The prior art is deficient in the lack of a method for using S2-6 gene products as a diagnostic marker for early cancer detection. The present invention fulfills this longstanding need and desire in the art.

### SUMMARY OF THE INVENTION

One embodiment of the present invention provides a method of detecting a cancer or a pre-cancer in a subject comprising the steps of obtaining a first tissue sample of interest from the subject, where the first sample is suspected of being cancerous or pre-cancerous; obtaining a second control tissue sample from the subject, where the second sample comprises healthy tissue from the tissue of interest as in the first sample; contacting the first tissue sample and the second tissue sample with an antibody or antibody fragment thereof, such that the antibody or antibody fragment specifically binds to an epitope of a S2-6 DNA binding protein; and comparing the amount of antibody or antibody fragment bound by the first sample with the amount of antibody or antibody fragment bound by the second sample; where no binding or a lesser binding of the antibody or antibody fragment by the first sample as compared to the amount of binding by the second sample is indicative of a cancer or a pre-cancer in the subject.

In another embodiment of this invention there is provided a method of detecting a cancer or a pre-cancer in a subject comprising the steps of obtaining a first tissue sample of interest from the subject, where the first sample is suspected of being cancerous or pre-cancerous; obtaining a second control tissue sample from the subject, where the second sample comprises healthy tissue from the tissue of interest as in the first sample; isolating S2-6 mRNA from the first sample and from the second sample; amplifying the S2-6 mRNA from the first sample and from the second sample to form S2-6 cDNA for each of the samples; amplifying the S2-6 cDNA from the first sample and from the second sample where the amplification uses an oligonucleotide probe comprising a fluorescent reporter and a fluorescent quencher; monitoring the level of fluorescence emitted from the fluorescent reporter in the first sample and in the second sample during amplification of each of the first and second samples; positively correlating the level of fluorescence of the first reporter with the quantity of S2-6 mRNA in the first sample and the level of fluorescence of the second reporter with the quantity of S2-6 mRNA in the second sample; and comparing the quantity of S2-6 mRNA in the first sample with the amount of S2-6 mRNA in the second sample; where no mRNA or a lesser quantity of mRNA in the first sample as compared to the quantity of mRNA in the second sample is indicative of cancer or pre-cancer in the subject.

In yet another embodiment of this invention there is provided a kit to detect cancer or pre-cancer in a subject comprising an antibody or fragment thereof, where the antibody or antibody fragment specifically binds to an epitope of an S2-6 DNA binding protein; means for performing and quantitating the results of an assay determining the amount of antibody or antibody fragment binding to the epitope; and instructions for use of the kit.

In still another embodiment of the present invention there is provided a kit to detect a cancer or a pre-cancer in a subject comprising a forward primer, a reverse primer and an oligonucleotide probe, where the primers and the probe hybridizes to a polynucleotide having SEQ ID NO: 1 or the complement thereof and where said probe further comprising a fluorescent reporter molecule and a fluorescent quencher molecule; means for performing and quantitating the results of a real-time PCR; and instructions for use of the kit.

Other and further aspects, features, and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others that will become clear, are attained and can be understood in detail, more particular descriptions of the invention briefly summarized above may be had by reference to certain embodiments thereof that are illustrated in the appended drawings. These drawings form a part of the specification. It is to be noted, however, that the appended drawings illustrate preferred embodiments of the invention and therefore are not to be considered limiting in their scope.

FIGS. 1A-1C depict the DNA sequence of the S2-6 gene (SEQ ID NO: 1) and the amino acid sequence of the protein (SEQ ID NO: 2) containing the zinc binding LIM domain of S2-6.

FIG. 1D depicts the amino acid sequence of the LIM domain of the S2-6 protein (SEQ ID NO: 3).

FIG. 2A depicts a Western blot analysis of S2-6 protein expression in eight paired normal (N) and tumor (T) human colon biopsies.

FIG. 2B depicts a non-parametric Mann-Whitney statistical test (two-tailed P value=0.0137) of S2-6 protein expression in paired normal and tumor colon biopsies (n=8).

### DETAILED DESCRIPTION OF THE INVENTION

One embodiment of the present invention provides a method of detecting a cancer or a pre-cancer in a subject comprising the steps of obtaining a first tissue sample of interest from the subject, where the first sample is suspected of being cancerous or pre-cancerous; obtaining a second control tissue sample from the subject, where the second sample comprises healthy tissue from the tissue of interest as in the first sample; contacting the first tissue sample and the second tissue sample with an antibody or antibody fragment thereof, such that the antibody or antibody fragment specifically binds to an epitope of a S2-6 DNA binding protein; and comparing the amount of antibody or antibody fragment bound by the first sample with the amount of antibody or antibody fragment bound by the second sample; where no binding or a lesser binding of the antibody or antibody fragment by the first sample as compared to the amount of binding by the second sample is indicative of a cancer or a pre-cancer in the subject.

In this embodiment the S2-6 DNA binding protein has the amino acid sequence comprising SEQ ID NO: 2 or SEQ ID NO: 3, preferably SEQ ID NO: 3. The antibody may be a monoclonal antibody or a polyclonal antibody. Additionally, the antibody or antibody fragment may be linked to a detectable label. A representative example of a detectable cancers in the instant invention is colon cancer.

In an aspect of this embodiment the method further comprises administering to the patient a viral vector comprising S2-6 DNA having the nucleotide sequence identified in SEQ ID NO: 1; and repeating the detection method to determine if the amount of S2-6 antibody or antibody fragment thereof bound in the first sample has increased relative to the amount bound in the second sample where the increase indicates a therapeutic effect against the cancer or the pre-cancer. This aspect of this embodiment may be used to treat a patient having cancer.

In another embodiment of this invention there is provided a method of detecting a cancer or a pre-cancer in a subject comprising the steps of obtaining a first tissue sample of interest from the subject, where the first sample is suspected of being cancerous or pre-cancerous; obtaining a second control tissue sample from the subject, where the second sample comprises healthy tissue from the tissue of interest as in the first sample; isolating S2-6 mRNA from the first sample and from the second sample; amplifying the S2-6 mRNA from the first sample and from the second sample to form S2-6 cDNA for each of the samples; amplifying the S2-6 cDNA from the first sample and from the second sample where the amplification uses an oligonucleotide probe comprising a fluorescent reporter and a fluorescent quencher; monitoring the level of fluorescence emitted from the fluorescent reporter in the first sample and in the second sample during amplification of each of the first and second samples; positively correlating the level of fluorescence of the first reporter with the quantity of S2-6 mRNA in the first sample and the level of fluorescence of the second reporter with the quantity of S2-6 mRNA in the second sample; and comparing the quantity of S2-6 mRNA in the first sample with the amount of S2-6 mRNA in the second sample; where no mRNA or a lesser quantity of mRNA in the first sample as compared to the quantity of mRNA in the second sample is indicative of cancer or pre-cancer in the subject.

In this embodiment the oligonucleotide probe hybridizes to an S2-6 polynucleotide having at least that part of SEQ ID NO: 1 that encodes at least part of SEQ ID NO: 2 or SEQ ID NO: 3, preferably SEQ ID NO: 3. Representative cancers are those disclosed supra.

In an aspect of this embodiment the method further comprises administering to the patient a viral vector comprising S2-6 DNA having the nucleotide sequence identified in SEQ ID NO: 1; repeating the method steps to determine if the S2-6 mRNA level in the first sample has increased relative to the level in the second sample where the increase indicates a therapeutic effect against the cancerous condition. This aspect of this embodiment may be used to treat a patient having cancer.

In yet another embodiment of this invention there is provided a kit to detect cancer or pre-cancer in a subject comprising an antibody or fragment thereof, where the antibody or antibody fragment specifically binds to an epitope of an S2-6 DNA binding protein; means for performing and quantitating the results of an assay determining the amount of antibody or antibody fragment binding to the epitope; and instructions for use of the kit. The kit may further comprise a detectable label linked to the antibody. The antibodies and S2-6 DNA binding proteins are as disclosed supra.

In still another embodiment of the present invention there is provided a kit to detect a cancer or a pre-cancer in a subject comprising a forward primer, a reverse primer and an oligonucleotide probe, where the primers and the probe hybridizes to a polynucleotide having SEQ ID NO: 1 or the complement thereof and where said probe further comprising a fluorescent reporter molecule and a fluorescent quencher molecule; means for performing and quantitating the results of a real-time PCR; and instructions for use of the kit. The oligonucleotide probes and polynucleotides are as disclosed supra.

As used herein, “S2-6” refers to the human S2-6 gene and products encoded by the gene, preferably having all or partial sequences of any of SEQ ID NOS: 1-3.

As used herein, the term “cDNA” shall refer to the DNA copy of the mRNA transcript of a gene.

As used herein “specifically binding” or “specifically binds” refers to an antibody or antigenic fragment thereof that binds to the S2-6 protein at a level detectable within an immunoassay, such as ELISA, and does not react at a detectable level with unrelated proteins. Antibody or antigenic fragment binding is a noncovalent association.

As used herein, “vector” refers to a replicable nucleic acid construct, e.g., a plasmid or viral nucleic acid. Vectors may be used to amplify and/or express nucleic acid encoding the S2-6 protein. An expression vector is a replicable construct in which a nucleic acid sequence encoding a polypeptide is operably linked to suitable control sequences capable of effecting expression of the polypeptide in a cell. The need for such control sequences will vary depending upon the cell selected and the transformation method chosen. Generally, control sequences include a transcriptional promoter and/or enhancer, suitable mRNA ribosomal binding sites, an d sequences which control the termination of transcription and translation. Methods which are well known to those skilled in the art can be used to construct expression vectors containing appropriate transcriptional/translational control signals. See for example, the techniques described in Sambrook et al., 1989, Molecular Cloning: A Laboratory Manual (2nd Edition), Cold Spring Harbor Press, N.Y., which are incorporated by reference. A gene and its transcription control sequences are defined as being “operably linked” if the transcription control sequences effectively control the transcription of the gene. Vectors may be, but are not limited to, plasmid vectors and viral vectors. Examples of viral vectors are those derived from retroviruses, adenovirus and adeno-associated virus.

As used herein, the term “PCR” refers to the polymerase chain reaction that is the subject of U.S. Pat. Nos. 4,683,195 and 4,683,202 to Mullis, as well as other improvements now known in the art.

As used herein “quantitative real-time PCR” refers to a polymerase chain reaction which is monitored, usually by fluorescence, over time during the amplification process, to measure a parameter related to the extent of amplification of a particular sequence. The amount of fluorescence released during the amplification cycle is proportional to the amount of product amplified in each PCR cycle.

As used herein, “oligonucleotide” refers to the probe of the present invention and is defined as a molecule comprised of two or more ribonucleotides, preferably more than three. Its exact size will depend upon many factors which, in turn, depend upon the ultimate function and use of the oligonucleotide.

As used herein “subject” refers to a warm-blooded animal, preferably a human. The subject may be in a non-cancerous, a pre-cancerous or a cancerous condition.

Provided herein are diagnostic methods for the early detection of a cancer that is based on the comparison of the level of S2-6 expression in the suspected cancerous lesion with the level in the healthy neighboring tissue. As these diagnostic tests determine the absence of or the reduction of expression of a gene over-expressed in normal cells and is, therefore, not dependent on a cancer-specific marker, it is applicable to various suspected cancerous lesions, e.g., colon cancer. The decreased presence of S2-6 mRNA or protein in the suspected cancer lesion relative to that in nearby healthy tissue is indicative of a pre-cancerous or of a cancerous state in the subject. Generally, the subject is any warm-blooded animal, preferably a human.

In the instant invention S2-6 protein expression or lack thereof is immuno-determined with monoclonal or polyclonal antibodies that specifically binds to S2-6 protein. Generally, standard protocols for monoclonal and polyclonal antibody production known to those skilled in this art may be employed. Methods for preparation of polyclonal antisera are taught in, for example, Ausubel, F. M. et al., Current Protocols in Molecular Biology, Volume 2, pp. 11.12.1-11.12.9, John Wiley & Sons, Inc., 1997. Preparation of monoclonal antibodies is taught in, for example, Ausubel, F. M. et al., Current Protocols in Molecular Biology, Volume 2, pp. 11.4.1-11.11.5, John Wiley & Sons, Inc., 1997.

For example, polyclonal antibodies can be generated by using S2-6, or a fragment thereof, as an immunogen in an animal such as a rabbit. The immunogen is injected into the host animal using a schedule preferably incorporating one or more booster immunizations. The animal is bled periodically and the polyclonal antibodies may be isolated and purified from the antisera by such standard methods as affinity chromatography.

Methods known to those of skill in the art can be used to generate fragments of the S2-6 protein, e.g., by enzymatic digestion of naturally occurring or recombinant S2-6 protein, by recombinant DNA techniques using an expression vector that encodes a defined fragment of S2-6, or by chemical synthesis. The S2-6 protein, polypeptide or fragment thereof may be covalently linked to another polypeptide to increase antigenicity. For such procedures the DNA and its products are encoded at least in part by portions of SEQ ID NO: 1.

Monoclonal antibodies can be generated by using the S2-6 protein, preferably the zinc binding LIM domain of S2-6, the amino acid sequence of which is not homologous to any other known proteins, to immunize a laboratory animal such as a mouse. An immortal cell line derived from the immunized host animal that produces antibodies capable of specifically binding with S2-6 is prepared. Such monoclonal antibodies may be isolated from the supernatant of growing hybridoma colonies and, optionally, the yield of such antibodies enhanced, by known methods. Hybridomas highly reactive with and specific to the zinc binding LIM domain having SEQ ID NO: 3 are preferred.

S2-6 protein expression is detected in a tissue sample obtained from a subject suspected of having cancer by contacting the sample with the labeled antibody, e.g., radioactively tagged antibody specific for S2-6, and detecting the S2-6 protein using standard immunoassay techniques such as an enzyme-linked immunosorbent assay (ELISA). Antibody binding to the tissue sample indicates that the sample contains a component that specifically binds to an epitope within S2-6, preferably to part of the protein having at least part of SEQ ID NO: 2 and more preferably to the zinc binding LIM domain having SEQ ID NO: 3. Lack of binding correlates with lack of expression of the S2-6 marker and, thus, would be indicative of highly proliferating cells, such as cancerous or pre-cancerous cells.

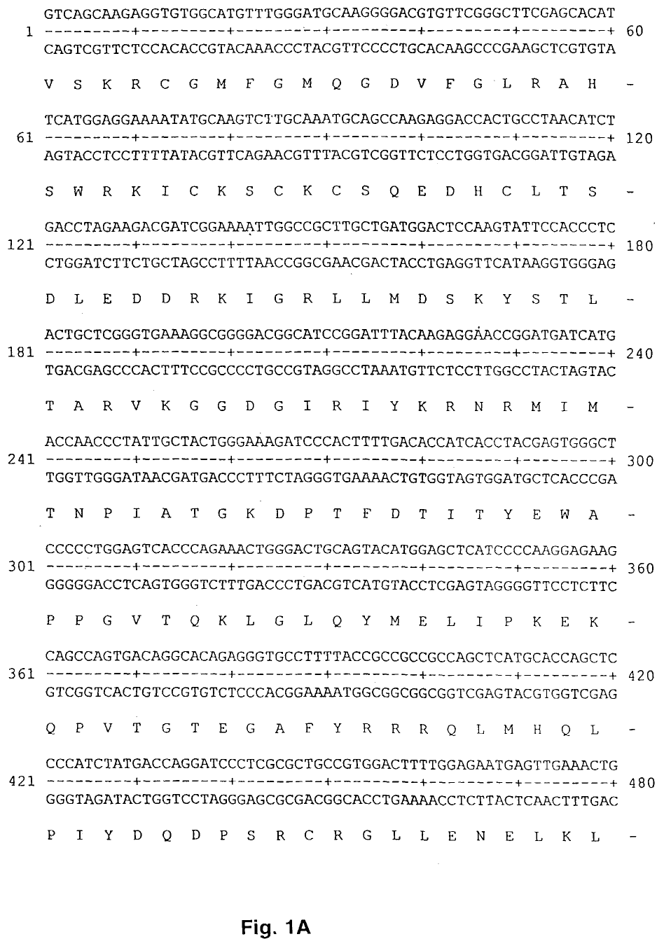

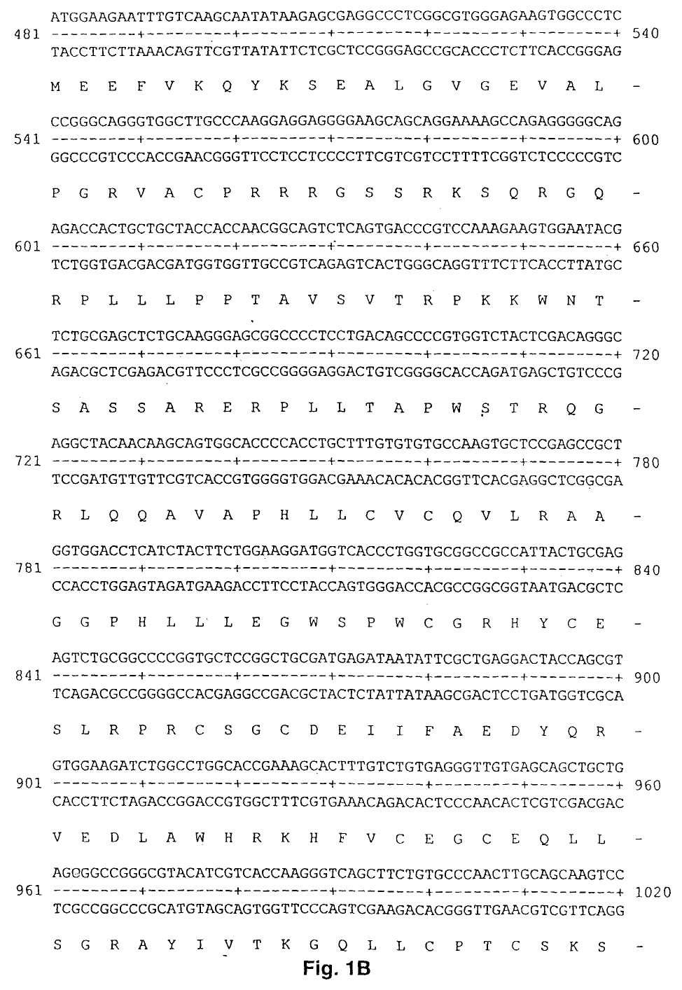

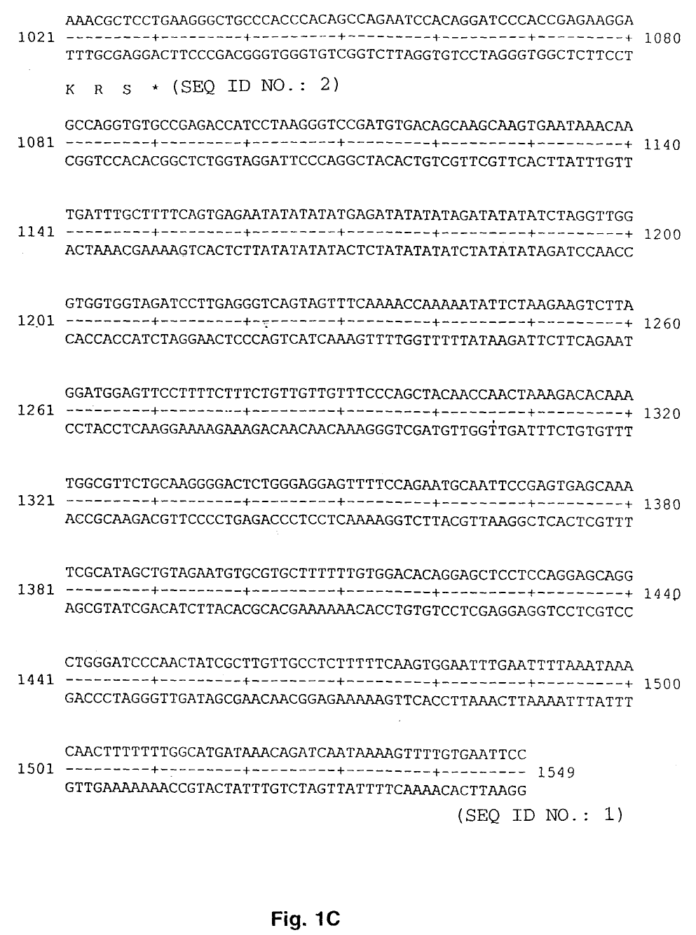

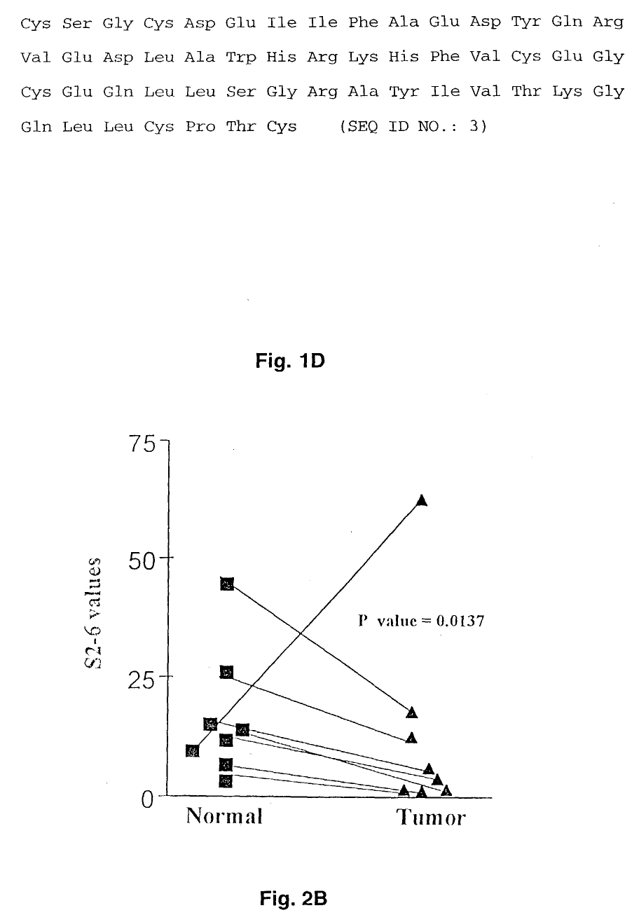

The zinc binding LIM domain is one to three repeats of a 52-residue segment containing two adjacent zinc binding domains separated by a two-residue linker (CX₂CX₁₇HX₂C)—X₂—(CX₂CX₁₇CX₂C/H/D). This amino acid sequence corresponds to bases 856 to 1011 of human S2-6 DNA of SEQ ID NO: 1 and to amino acids 286 to 337 of SEQ ID NO: 2 encoded by the DNA (FIG. 1A). Preferably, this antibody specifically binds to an epitope in the zinc binding LIM domain of the S2-6 protein corresponding to amino acid sequence SEQ ID NO: 3: C S G C D E I I F A E D Y Q R V E D L A W H R K H F V C E G C E Q L L S G R A Y I V T K G Q L L C P T C (FIG. 1B).

The antibody encompasses not only an intact monoclonal antibody, but also an immunologically-active antibody fragment, e.g., a Fab or (Fab)₂ fragment, an engineered single chain Fv molecule or a chimeric molecule, such as an antibody which contains the binding specificity of one antibody, for example, one of murine origin, with the remaining portions being that of another antibody, for example, those of human origin. Purified S2-6 or antigenic fragments of S2-6 can be used to test existing antibodies as positive controls in the diagnostic assay by employing standard protocols known to those skilled in the art. Tissue samples can be, for example, cells, blood, plasma, tissue, etc.

The antibody, or a fragment thereof, may be linked to a toxin or to a detectable label, e.g. a radioactive label, non-radioactive isotopic label, fluorescent label, chemiluminescent label, paramagnetic label, enzyme label, or colorimetric label. Examples of suitable toxins include diphtheria toxin, Pseudomonas exotoxin A, ricin, and cholera toxin. Examples of suitable enzyme labels include malate hydrogenase, staphylococcal nuclease, delta-5-steroid isomerase, alcohol dehydrogenase, alpha-glycerol phosphate dehydrogenase, triose phosphate isomerase, peroxidase, alkaline phosphatase, asparaginase, glucose oxidase, beta-galactosidase, ribonuclease, urease, catalase, glucose-6-phosphate dehydrogenase, glucoamylase, acetylcholinesterase, etc. Examples of suitable radioisotopic labels include ³H, ¹²⁵I, ¹³¹I, ³²P, ³⁵S, ¹⁴C, etc.

Examples of suitable fluorescent labels include a fluorescein label, an isothiocyalate label, a rhodamine label, a phycoerythrin label, a phycocyanin label, an allophycocyanin label, an ophthaldehyde label, a fluorescamine label, etc. Examples of chemiluminescent labels include a luminal label, an isoluminal label, an aromatic acridinium ester label, an imidazole label, an acridinium salt label, an oxalate ester label, a luciferin label, a luciferase label, an aequorin label, etc.

Those of ordinary skill in the art will know of other suitable labels that may be employed in accordance with the present invention. The binding of these labels to antibodies or fragments thereof can be accomplished using standard techniques commonly known to those of ordinary skill in the art. Typical techniques are described by Kennedy et al., (1976) Clin. Chim. Acta 70, 1-31; and Schurs et al., (1977) Clin. Chim. Acta 81, 1-40. Coupling techniques mentioned in the latter are the glutaraldehyde method, the periodate method, the dimaleimide method, the m-maleimidobenzyl-N-hydroxy-succinimide ester method.

S2-6 mRNA expression in a tissue sample obtained from a subject suspected of having cancer may be determined; the level of expression is indicative of the presence or absence of a cancerous. Polynucleotide primers and probes may be used to detect the level mRNA encoding S2-6 in a quantitative real time PCR assay. Preferably these primers and probes hybridize to a strand of DNA having some or all of SEQ ID NO: 1 or the complement thereof. To determine S2-6 mRNA expression, S2-6 mRNA is first amplified to form cDNA using a standard reverse transcription PCR. The advantage of quantitative real-time PCR over conventional PCR is that product is quantitated during amplification and not after. The sensitivity of real-time PCR is such that quantitation may be carried out while amplification is still in the log phase, requiring as few as five amplification cycles and thus providing high throughput.

In real-time PCR an oligonucleotide probe, that anneals specifically between the forward and reverse PCR primers, contains two fluorochromes, i.e., a fluorescent reporter and a quencher. A reporter fluorochrome, such as 6-carboxyfluorescein (6-FAM), JOE or VIC, is placed on the 5′ end of a probe and a quencher fluorochrome, such as 6-carboxy-tetramethyl-rhodamine (TAMRA), is placed on the 3′ end of the probe or, alternatively, at any T position on the probe. During the extension phase of the PCR, the probe, i.e., TaqMan, which is designed to have a higher Tₘ than the primers, must be 100% hybridized. While both of the fluorochromes and the probe are intact, the quencher prevents the reporter from fluorescing. During amplification, however, as the Taq polymerase extends the primer, the inherent 5′ exonuclease activity of the polymerase cleaves the probe and releases the reporter which, since it is now free from the quencher, generates a sequence specific fluorescent signal. With each cycle, additional reporter dye molecules are cleaved from their probes and the fluorescence intensity can be monitored at regular intervals.

Additionally, S2-6 expression, either mRNA or protein, is analyzed in situ on histological tissue sections. Monitoring the proportion of cells that do not express the S2-6 gene product, together with analysis of morphological appearance and expression of other tumor markers, allow for simultaneous identification of the cell type that undergoes cancerous transformation and the stage of the disease.

Thus, in addition to using S2-6 as an early marker for cancer detection, the S2-6 gene may also be used a marker for effectiveness of cancer treatment. Sequential determination of an increase in S2-6 protein and/or mRNA expression in cancerous tissue or cells would indicate a reduction in proliferation of tumor cells. Monitoring the number of immortal or cancerous cells in tumor lesions during the pharmacological therapy or after tumor resection is an indicator of the individual's progress. This may be done as frequently as determined is necessary after commencement of therapy or after resection and then performed intermittently as needed thereafter. Generally, a cancer is not progressing in those subjects in whom the level of S2-6 expression increases or at least remains constant.

Moreover, it is contemplated that the S2-6 gene can provide a method of cancer therapy, such as gene replacement therapy, for patients bearing lesion(s) which display lost or reduced S2-6 expression. As the S2-6 gene sequence could play a role in DNA replication, cell growth and differentiation, causing overexpression of the S2-6 mRNA in cancer cells could inhibit cell growth. The transfection of cancerous cells with a retroviral vector comprising and expressing the S2-6 gene, for example, allows for the constant overexpression of S2-6 mRNA and protein in these cancer cells. The vector contemplated contains a DNA having the nucleotide sequence identified in SEQ ID NO: 1 that encodes a recombinant polypeptide that includes at least the amino acid sequence having SEQ ID NO: 2.

A screening method for an individual suspected of having cancer or predisposed to a cancerous condition is also contemplated herein. As the S2-6 gene is a non-specific cancer marker, a comprehensive tissue microarray-based immunohistochemical and/or in situ hybridization test can be conducted to screen for a variety of tumor types of those tissues known to normally express S2-6 gene products. As a decrease in S2-6 expression correlates with an increase in the proliferative state of a cell and, by extension, a potential neoplastic state, an individual can be monitored by comparing the amount of S2-6 expression to his/her own internal standard.

The methodology based on the comparison of the level of S2-6 mRNA and/or protein in suspected cancer lesions and equivalent normal tissue provides the basis for an easy, fast and relatively inexpensive test for in vitro or in vivo early cancer detection and/or monitoring. A kit to conduct such tests is provided herein. The kit may comprise at least two components needed to perform a diagnostic assay. These may be compounds, reagents, containers and/or equipment; optionally the containers may contain, for example, reagents or buffers used in the assay.

Further, it is contemplated that the kits may contain at least one component that is frozen, lyophilized, pre-diluted, or pre-mixed at such pre-determined concentrations that the addition of a specific amount of heat, of water or of a solution, optionally provided in the kit, will reconstitute the components into an effective active composition. For example, such an active composition may be at the required concentration or have the required pH for immediate use. A kit may contain a reagent, e.g., a reporter group, used to detect, either directly or indirectly, antibody binding or gene expression. An example of such a reporter group is a fluorophore or radiolabeled component as previously disclosed. Preferably, such a kit will also comprise instructions for reconstituting and using the active compositions to detect cancer. The above-noted buffers, reagents, and other component parts can be sold separately or together with the kit.

In one example a kit may contain a monoclonal antibody or fragment thereof that specifically binds to the S2-6 protein having SEQ ID NO: 2 or a portion thereof, particularly the zinc binding LIM domain having SEQ ID NO: 3. These antibodies or fragments may b e frozen or lyopholized and may be prepared for use by, respectively, thawing and optionally further diluting the monoclonal antibodies in a suitable buffer, for example, or by reconstituting the antibody in a liquid, e.g., a buffer. Alternatively, the antibodies may be attached to a support material as is known in the art. Other components that may be included within such a kit are diagnostic reagents or containers to facilitate the detection of antibody binding to the S2-6 protein or fragment thereof. Such components may comprise the means to perform and to quantitate the results of an ELISA assay.

In another example a kit may contain those components used to detect the presence and/or level of mRNA encoding the S2-6 protein in a biological sample. These may comprise at least one oligonucleotide probe or primer, as described above, that hybridizes to a polynucleotide encoding the S2-6 protein or fragment thereof. Such a polynucleotide may be S2-6 mRNA or the cDNA thereof specifically having some or all of SEQ ID NO: 1 or the complement thereof. Other components that may be included within such a kit are a second oligonucleotide and/or a diagnostic reagent or container to facilitate the detection of the polynucleotide encoding the S2-6 protein or fragment thereof. Such components may comprise the means to perform and to quantitate the results of real-time PCR.

The following examples are given for the purpose of illustrating various embodiments of the invention and are not meant to limit the present invention in any fashion.

### EXAMPLE 1

S2-6 Protein Expression in Paired Normal and Tumor Human Colon Biopsies.

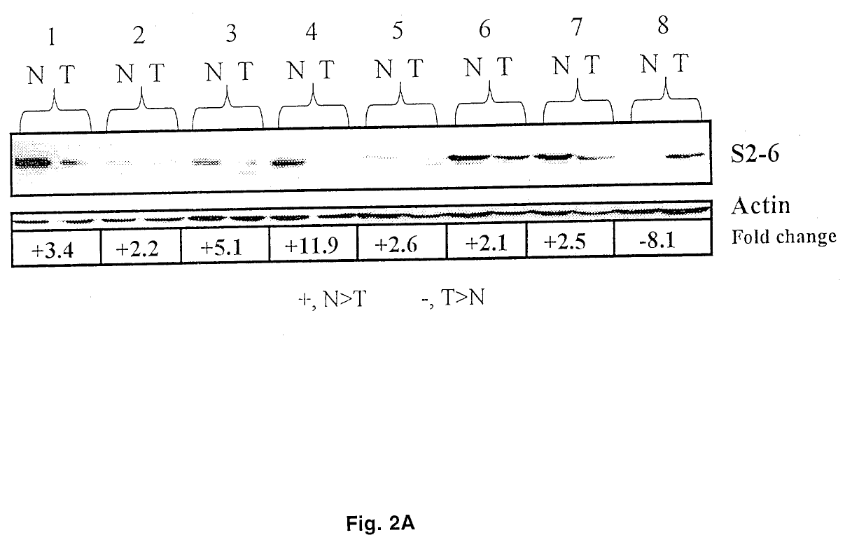

Western blot analysis of S2-6 protein expression in paired normal (N) and tumor (T) human colon biopsies from eight different patients is shown in FIG. 2A. 40 μg aliquots of tumor/normal protein extracts are separated on a 12% SDS-PAGE gel. Blots are then probed with anti-human S2-6 polyclonal antibody. The level of S2-6 expression is expressed as a function of actin protein expression; this data is used to calculate the fold differences between normal and tumor tissue samples from the same patient. FIG. 2B shows an analysis of the paired data (n=8) using the non-parametric Mann-Whitney statistical test (two-tailed P value=0.0137).

With the exception of patient 8 where expression was significantly higher in tumor tissue, S2-6 protein expression was a t least 2-fold higher in normal colon tissue. In those seven patients fold increases ranged from +2.1 to +11.9. The disparity in the fold differences from patient to patient highlights the benefits of detecting or monitoring S2-6 protein expression by using the patients own internal standard. Thus, a comparison of S2-6 protein expression at any point is relative to the status of the patient and is not based on a generalized standard curve.

The following references are cited herein:

1. Noda et al. Exp. Cell Res. Vol. 211, pp. 90-98 (1994).

2. Xiong et al. Nature, Vol. 366, pp. 701-704 (1993.).

3. Harper et al. Cell, Vol. 75, pp. 805-816 (1993).

4. Liu et al. Cancer Lett. Vol. 170, pp. 183-189 (2001).

5. Gohring et al. J. Clin. Path. Vol. 54, pp. 866-870 (2001).

6. Elkablawy et al. J. Path. Vol. 194, pp. 436-443 (2001).

Any patents or publications mentioned in this specification are indicative of the levels of those skilled in the art to which the invention pertains. These patents and publications are herein incorporated by reference to the same extent as if each individual publication was indicated to be incorporated specifically and individually by reference.

One skilled in the art will readily appreciate that the present invention is well adapted to carry out the objects and obtain the ends and advantages mentioned, as well as those inherent therein. It will be apparent to those skilled in the art that various modifications and variations can be made in practicing the present invention without departing from the spirit or scope of the invention. Changes therein and other uses will occur to those skilled in the art which are encompassed within the spirit of the invention as defined by the scope of the claims.

What is claimed is:

1. A method of detecting a cancer or a pre-cancer in a subject comprising the steps of:
a) obtaining a first tissue sample of interest from the subject, said first sample suspected of being cancerous or pre-cancerous;
b) obtaining a second control tissue sample from the subject, said second sample comprising healthy tissue from the tissue of interest as in said first sample;
c) contacting said first tissue sample and said second tissue sample with an antibody or antibody fragment thereof, said antibody or antibody fragment specifically binding to an epitope of a S2-6 DNA binding protein; and
d) comparing the amount of antibody or antibody fragment bound by said first sample with the amount of antibody or antibody fragment bound by said second sample; wherein no binding or a lesser binding of said antibody or antibody fragment by said first sample as compared to the amount of binding by said second sample is indicative of said cancer or said pre-cancer in said subject.

2. The method of claim 1, further comprising the steps of:
i) administering to said patient a viral vector comprising S2-6 DNA having the nucleotide sequence identified in SEQ ID NO: 1;
j) repeating steps a) to d) to determine if the amount of S2-6 antibody or antibody fragment thereof bound in said first sample has increased relative to the amount bound in said second sample wherein said increase indicates a therapeutic effect against said cancer or said pre-cancer.

3. The method of claim 1, wherein said antibody is a monoclonal antibody or a polyclonal antibody.

4. The method of claim 3, wherein said method is a means of treating said cancer in said subject.

5. The method of claim 1, wherein said S2-6 DNA binding protein has the amino acid sequence comprising SEQ ID NO: 2 or SEQ ID NO: 3.

6. The method of claim 5, wherein said S2-6 DNA binding protein has the amino acid sequence comprising SEQ ID NO: 3.

7. The method of claim 1, wherein said antibody or said antibody fragment is linked to a detectable label.

8. The method of claim 1, wherein said cancer is colon cancer.

9. A method of detecting a cancer or a pre-cancer in a subject comprising the steps of:
a) obtaining a first tissue sample of interest from the subject, said first sample suspected of being cancerous or pre-cancerous;
b) obtaining a second control tissue sample from the subject, said second sample comprising healthy tissue from the tissue of interest as in said first sample;
c) isolating S2-6 mRNA from said first sample and from said second sample;
d) amplifying said S2-6 mRNA from said first sample and from said second sample to form S2-6 cDNA for each of said samples;
e) amplifying said S2-6 cDNA from said first sample and from said second sample wherein said amplification uses an oligonucleotide probe comprising a fluorescent reporter and a fluorescent quencher;
f) monitoring the level of fluorescence emitted from the fluorescent reporter in said first sample and in said second sample during amplification of each of said first and said second samples;
g) positively correlating the level of fluorescence of said first reporter with the quantity of S2-6 mRNA in said first sample and the level of fluorescence of said second reporter with the quantity of S2-6 mRNA in said second sample; and
h) comparing the quantity of S2-6 mRNA in said first sample with the amount of S2-6 mRNA in said second sample; wherein no mRNA or a lesser quantity of mRNA in said first sample as compared to the quantity of mRNA in said second sample is indicative of said cancer or said pre-cancer in said subject.

10. The method of claim 9, further comprising the steps of:
i) administering to said patient a viral vector comprising S2-6 DNA having the nucleotide sequence identified in SEQ ID NO: 1;
j) repeating steps a) to h) to determine if the S2-6 mRNA level in said first sample has increased relative to the level in said second sample wherein said increase indicates a therapeutic effect against said cancerous condition.

11. The method of claim 10, wherein said method is a means of treating said cancer in said subject.

12. The method of claim 10, wherein said oligonucleotide probe hybridizes to an S2-6 polynucleotide having a t least that part of SEQ ID NO: 1 that encodes at least part of SEQ ID NO: 2 or SEQ ID NO: 3.

13. The method of claim 12, wherein said S2-6 polynucleotide encodes at least part of SEQ ID NO: 3.

14. The method of claim 10, wherein said cancer is colon cancer.

15. A kit to detect a cancer or a pre-cancer in a subject comprising:
a) an antibody or fragment thereof, said antibody or antibody fragment specifically binding to an epitope of an S2-6 DNA binding protein;
b) means for performing and quantitating the results of an assay determining the amount of antibody or antibody fragment binding to said epitope; and
c) instructions for use of said kit.

16. The kit of claim 15, further comprising a detectable label linked to said antibody or antibody fragment.

17. The kit of claim 15, wherein said antibody is a monoclonal antibody or a polyclonal antibody.

18. The kit of claim 15, wherein said S2-6 DNA binding protein has the amino acid sequence comprising SEQ ID NO: 2 or SEQ ID NO: 3.

19. The kit of claim 18, wherein said S2-6 DNA binding protein has the amino acid sequence comprising SEQ ID NO: 3.

20. A kit to detect a cancer or a pre-cancer in a subject comprising:
a) a forward primer, a reverse primer and an oligonucleotide probe, said primers and said probe hybridizing to a polynucleotide having SEQ ID NO: 1 or the complement thereof; said probe further comprising a fluorescent reporter molecule and a fluorescent quencher molecule;
b) means for performing and quantitating the results of a real-time PCR; and
c) instructions for use of said kit.

21. The kit of claim 20, wherein said oligonucleotide probe hybridizes to an S2-6 polynucleotide having at least that part of SEQ ID NO: 1 that encodes at least part of SEQ ID NO: 2 or SEQ ID NO: 3.

22. The kit of claim 20, wherein said S2-6 polynucleotide encodes at least part of SEQ ID NO: 3.
